(19) United States
(12) Patent Application Publication
(10) Pub. No.: US 2009/0239803 A1
(43) Pub. Date: Sep. 24, 2009

(54) BITE-SIZE CARBOHYDRATE NUTRITIONAL PRODUCTS AND METHODS FOR USING SAME

(75) Inventors: Marie Georgina Feuvrier-Roy, Chaux-Neuve (FR); Donald Joseph Leonard, Los Angeles, CA (US); Ankur Mittal, Lutry (CH); Asker Jeukendrup, Birmingham West Midlands (GB); Trent Stellingwerff, Le Mont-Sur-Lausanne (CH); Eric Zaltas, St-Saphorin (CH)

Correspondence Address: K&L Gates LLP, P.O. Box 1135, CHICAGO, IL, 60690, US

(73) Assignee: NESTEC S.A., Vevey (CH)

(21) Appl. No.: 12/396,723

(22) Filed: Mar. 3, 2009

Related U.S. Application Data
(60) Provisional application No. 61/088,559, filed on Aug. 13, 2008.

(30) Foreign Application Priority Data
Mar. 3, 2008 (EP) 08152222.9

(51) Int. Cl. A61K 38/00; A23L 1/09; A61K 31/7004; A61P 43/00

(52) U.S. Cl. 514/12; 426/658; 514/25

(57) ABSTRACT

Bite-size carbohydrate-based products and methods of using same are provided. In a general embodiment, the present disclosure provides a bite-size carbohydrate-based product comprises a carbohydrate source comprising a glucogenic:fructogenic carbohydrate ratio ranging between about 1.5 to about 2.5 with at least 60% of the energy content of the bite-size carbohydrate-based product coming from the carbohydrate source. Along with the carbohydrate source, the bite-size carbohydrate-based product further comprises a suitable amount of sodium. The bite-size carbohydrate-based products can be specifically designed and provided to an athlete for customized carbohydrate intake and portion control before, during and/or after exercising.

### PRIORITY CLAIM

This application claims the benefit of U.S. Provisional Patent Application Ser. No. 61/088,559 filed on Aug. 13, 2008 and claims priority to European Patent Application EP 08152222.9 filed in the European Patent Office Mar. 3, 2008, the entire disclosures of which are hereby incorporated by reference in their entirety.

### BACKGROUND

The present disclosure generally relates to methods for providing nutrition before, during and/or after exercising. More specifically, the present disclosure is directed to bite-size carbohydrate-based products and methods for using same before, during and/or after exercising.

Nutritional products for providing energy to a consumer are known. Typically, these nutritional products are in the shape of large bars and are bulky to consume. Moreover, these nutritional products are not associated with any recommended intake requirements based on the types of consumers or activities of the consumers. For example, an athlete who engages in endurance activities will likely need to eat a greater amount of the nutritional product than a person going for a casual walk. As a result, a person who eats the nutritional products does not know how much of the nutritional product to consume in order to optimize the energy providing contents of the nutritional product.

### SUMMARY

The present disclosure provides bite-size carbohydrate-based products that can be used for customized carbohydrate intake before, during and/or after exercising. In a general embodiment, the present disclosure provides a bite-size carbohydrate-based product comprising a carbohydrate source comprising a glucogenic:fructogenic carbohydrate ratio ranging between about 1.5 to about 2.5 with at least 60% (e.g. in the form of calories) of the energy content of the bite-size carbohydrate-based product coming from the carbohydrate source. Along with the carbohydrate source, the bite-size carbohydrate-based product further comprises a suitable amount of sodium.

In an embodiment, the sodium ranges from about 200 mg to about 400 mg of sodium per 100 g of the bite-size carbohydrate-based product.

In an embodiment, the bite-size carbohydrate-based product further comprises a fat, a fiber and a protein.

In an embodiment, the bite-size carbohydrate-based product comprises an energy content of between about 30 to kcal about 45 kcal.

In an embodiment, the bite-size carbohydrate-based product further comprises a component such as oat flakes, whole crispies, natural fruits or a combination thereof.

In another embodiment, the present disclosure provides a method of providing nutrition to an athlete. The method comprises providing a bite-size carbohydrate-based product comprising a carbohydrate source comprising a glucogenic:fructogenic carbohydrate ratio ranging between about 1.5 to about 2.5 with at least 60% of the energy content of the bite-size carbohydrate-based product coming from the carbohydrate source. The bite-size carbohydrate-based product further comprises a suitable amount of sodium. The method further comprises providing personalized guidelines for consuming the bite-size carbohydrate-based product. The personalized guidelines provide the recommended amount of the bite-size carbohydrate-based product to consume before and during exercising based on at least one of a characteristic of the athlete, a training regimen of the athlete or any other type of sport or duration of activities.

In an embodiment, the characteristic of the athlete is weight, height, age, gender or a combination thereof.

In an embodiment, the training regimen is running, swimming, cycling, hiking, football, basketball, baseball, soccer or any other suitable individual or group sport.

In an embodiment, the personal guidelines are further based on the duration of the training regiment of the athlete.

In an embodiment, the training regimen is determined based on a sport.

In an embodiment, the training regimen is determined based on a personal objective.

In an embodiment, the personalized guidelines provide the recommended amount of the bite-size carbohydrate-based product to consume during exercising based on at least one of the characteristic of the athlete, a training regimen of the athlete or any other type of sport or duration of activities.

In an alternative embodiment, the present disclosure provides a method of selling a bite-size carbohydrate-based product. The method comprises providing a plurality of bite-size carbohydrate-based products in a package. The bite-size carbohydrate-based products comprise a carbohydrate source comprising a glucogenic:fructogenic carbohydrate ratio ranging between about 1.5 to about 2.5 with at least 60% of the energy content of the bite-size carbohydrate-based product coming from the carbohydrate source. The bite-size carbohydrate-based product further comprises a suitable amount of sodium. The method further comprises providing personalized guidelines for consuming the bite-size carbohydrate-based products. The personalized guidelines provide the recommended amount of the bite-size carbohydrate-based product to consume before and during exercising. The bite-size carbohydrate-based product package and the guidelines can then be sold to an athlete.

In an embodiment, the personalized guidelines are based on at least one of a characteristic of the athlete, a training regimen of the athlete or any other type of sport or duration of activities.

In an embodiment, the bite-size carbohydrate-based products are not individually wrapped.

In an embodiment, the bite-size carbohydrate-based products are individually wrapped.

In another embodiment, the present disclosure provides a nutritional kit comprising a plurality of bite-size carbohydrate-based products and personalized guidelines on how many of the bite-size carbohydrate-based products an athlete should consume before and during exercising. The bite-size carbohydrate-based products comprise a carbohydrate source comprising a glucogenic:fructogenic carbohydrate ratio ranging between about 1.5 to about 2.5 with at least 60% of the energy content of the bite-size carbohydrate-based product coming from the carbohydrate source. The bite-size carbohydrate-based product further comprises a suitable amount of sodium.

In an embodiment, a plurality of the bite-size carbohydrate-based products and the personalized guidelines are together in a package.

In an embodiment, the personalized guidelines provide the recommended amount of the bite-size carbohydrate-based product to consume before and during exercising based on a physical characteristic of the athlete.

In an embodiment, the personalized guidelines provide the recommended amount of the bite-size carbohydrate-based product to the athlete before and during exercising based on the training regimen of the athlete.

In an embodiment, the personalized guidelines provide the recommended amount of the bite-size carbohydrate-based product to consume before and during exercising based on the type of sport that the athlete engages in.

In an embodiment, the personalized guidelines provide the recommended amount of the bite-size carbohydrate-based product to the athlete before and during exercising based on the duration of the physical activities that the athlete engages in.

In an alternative embodiment, the present disclosure provides a method of enhancing the performance of an athlete. The method comprises providing a bite-size carbohydrate-based product comprising a carbohydrate source comprising a glucogenic:fructogenic carbohydrate ratio ranging between about 1.5 to about 2.5 with at least 60% of the energy content of the bite-size carbohydrate-based product coming from the carbohydrate source. The bite-size carbohydrate-based product further comprises a suitable amount of sodium. The method further comprises providing personalized guidelines for consuming the bite-size carbohydrate-based product. The personalized guidelines provide the recommended amount of the bite-size carbohydrate-based product to consume before the performance based on at least one of a characteristic of the athlete, a training regimen of the athlete or any other type of sport or duration of activities.

In another embodiment, the present disclosure provides a method of improving recovery of an athlete after exercising. The method comprises providing a bite-size carbohydrate-based product comprising a carbohydrate source comprising a glucogenic:fructogenic carbohydrate ratio ranging between about 1.5 to about 2.5 with at least 60% of the energy content of the bite-size carbohydrate-based product coming from the carbohydrate source. The bite-size carbohydrate-based product further comprises a suitable amount of sodium. The method further comprises providing personalized guidelines for consuming the bite-size carbohydrate-based product. The personalized guidelines provide the recommended amount of the bite-size carbohydrate-based product to consume during recovery after exercising based on at least one of a characteristic of the athlete and a training regimen of the athlete.

An advantage of the present disclosure to provide improved methods of providing bite-size carbohydrate-based products before, during and/or after exercising.

Another advantage of the present disclosure is to provide new methods for customized carbohydrate intake before, during and/or after exercising.

Still another advantage of the present disclosure is to provide an improved method for providing optimized energy to an athlete before, during and/or after exercising.

Additional features and advantages are described herein, and will be apparent from, the following Detailed Description.

### DETAILED DESCRIPTION

The present disclosure relates generally to methods for providing nutrition to an athlete before, during and/or after exercising. For example, bite-size carbohydrate-based products can be specifically designed and provided to an athlete for customized energy intake and portion control before, during and/or after exercising.

In a general embodiment, the present disclosure provides a bite-size carbohydrate-based product comprising a carbohydrate source comprising a glucogenic:fructogenic carbohydrate ratio ranging between about 1.5 to about 2.5 with at least 60% of the energy content of the bite-size carbohydrate-based product coming from the carbohydrate source. Along with the carbohydrate source, the bite-size carbohydrate-based product further comprises a suitable amount of sodium. The sodium can range in an amount from about 200 mg to about 400 mg of sodium per 100 g of the bite-size carbohydrate-based product. In an embodiment, each of the bite-size carbohydrate-based product comprises an energy content of between about 20 kcal to about 45 kcal.

It has been surprisingly found that a glucogenic:fructogenic carbohydrate ratio between about 1.5 and about 2.5 is optimum. When the ratio is within the cited range, it has been found that the availability of the energy (from carbohydrates) to the muscles is optimized. The glucidic sources of energy are quickly absorbed though the digestive tract and transported to the muscles. It is speculated, without being bound by the theory, that such a ratio provides for an optimum utilization of the glucose (respectively fructose) receptors and transporters. With both pathways (glucose and fructose) being utilized optimally, a beneficial effect is observed in the availability of the sources of energy to the muscles. Also, it is speculated that the energy required to absorb and transport the molecules to the muscles is comparatively lowered.

In one embodiment, the glucogenic:fructogenic carbohydrate ratio is between about 1.7 and about 2.3. In other embodiments, the glucogenic:fructogenic carbohydrate ratio is between about 1.8 and about 2.2, between about 1.9 and about 2.1. In another embodiment, the glucogenic:fructogenic carbohydrate ratio is 2.0 or about 2.0. All references to percentages are percentages by weight (of dry matter) unless otherwise stated.

The glucogenic:fructogenic carbohydrate ratio is the ratio obtained by dividing total number of glucose molecules over the total number of fructose molecules in a theoretical complete hydrolysis of all the carbohydrates present in the composition. For example, for a mix comprising 80% pure glucose and 20% pure fructose, the ratio of glucose/fructose=80/20=4. For a 100% high fructose corn syrup (high fructose corn syrup contains 45% glucose and 55% fructose), the ratio of glucose/fructose=45/55=0.8. For a 100% saccharose (=sucrose=50% fructogenic carbohydrate 50% glucogenic carbohydrate), the ratio of glucose/fructose=50/50=1. For a 50% saccharose and 50% corn syrup [100% glucogenic carbohydrate], the ratio of glucose/fructose=(50+100)/50=3.

The determination of the glucose and fructose carbohydrate content can be performed by any suitable conventional methods. For example, the total sugar can be determined by acid hydrolysis followed by a sugar profile analysis using chromatography.

The glucose and/or fructose may be provided in the form of fructogenic and/or glucogenic carbohydrates. In this specification, fructogenic carbohydrates are carbohydrates that upon theoretical total hydrolysis release at least one fructose molecule. Glucogenic carbohydrates are carbohydrates that upon theoretical total hydrolysis release at least one glucose molecule. Consequently, a carbohydrate can be both, glucogenic and fructogenic (e.g., saccharose). In the bite-size carbohydrate-based products, glucogenic sources can also in the form of extruded cereal pieces, cereal flakes, flours and starches.

The carbohydrates may comprise or consist of monosaccharides, such as glucose or fructose as basic carbohydrate units. The monosaccharides may represent a part of disaccharides, such as sucrose, lactose, maltose or cellobiose. The monosaccharides such as glucose or fructose may also represent a part of oligosaccharides or polysaccharides. Preferred carbohydrate sources for the present invention are maltodextrins and/or dextrose.

The carbohydrate source may additionally comprise indigestible carbohydrates, in particular fibers. The carbohydrate fraction may comprise at least 30% glucose and fructose, preferably at least 50% glucose and fructose, and more preferably at least 85% glucose and fructose. In one embodiment of the present disclosure, the carbohydrate fraction of the bite-size carbohydrate-based product provides at least 60%, preferably at least 70% of the energy of the bite-size carbohydrate-based product (e.g. in the form of calories).

The bite-size carbohydrate-based product can be in a bite-size form and comprise any suitable shape for ease of handling. For example, the bite-size carbohydrate-based product can be substantially in the shape of a sphere having a diameter ranging from about 12 mm to about 35 mm. Preferably, the diameter of the sphere ranges from about 15 mm to about 25 mm.

In an embodiment, the bite-size carbohydrate-based product weighs from about 5 grams to about 25 grams. Preferably, each bite-size carbohydrate-based product can comprise a weight ranging from about 5 grams to about 15 grams. More preferably, each bite-size carbohydrate-based product can comprise a weight ranging from about 8 grams to 9 grams.

The bite-size carbohydrate-based product can also comprise additional ingredients. For example, the bite-size carbohydrate-based product can comprise one or more fats, fibers and/or proteins. In another embodiment, the bite-size carbohydrate-based product further comprises oat flakes, whole crispies, natural fruits or a combination thereof. In an alternative embodiment, the bite-size carbohydrate-based product comprises one or more flavors and ingredients such as peanut butter, dark chocolate chips, oatmeal raisin, cranberry crunch, blueberry muesli, peach crumble, etc. The bite-size carbohydrate-based product can further comprise a crunchy texture.

The presence of proteins and/or fats in the bite-size carbohydrate-based product of the present disclosure has the advantage in that it is possible to provide an athlete with a more complete nutrition during performance. For the protein source, any suitable dietary protein may be such as, for example, animal proteins (e.g. milk proteins, meat proteins and egg proteins); vegetable proteins (e.g. soy protein, wheat protein, rice protein, and pea protein); mixtures of free amino acids; or combinations thereof. Milk proteins, such as casein and whey milk proteins, and soy proteins are particularly preferred.

The proteins may be intact or hydrolysed or a mixture of intact and hydrolysed proteins. It may be desirable to supply partially hydrolysed proteins (degree of hydrolysis between 2 and 20%), for example for athletes believed to be at risk of developing cows' milk allergy. Generally, at least partially hydrolysed proteins are easier and faster to metabolize by the body. This is in particular true for amino acids.

If the bite-size carbohydrate-based product includes a fat source, the fat source has the advantage in providing for an improved mouth feel. Any fat source is suitable. For example, animal or plant fats may be used. To increase the nutritional value, n3-unsaturated and n6-unsaturated fatty acids may comprise the fat source. The fat source may also contain long chain fatty acids and/or medium chain fatty acids. For example, milk fat, canola oil, cocoa butter, almond butter, peanut butter, corn oil and/or high-oleic acid sunflower oil may be used.

In another embodiment, the present disclosure provides a method of providing nutrition to an athlete using customized or personal guidelines in conjunction with the bite-size carbohydrate-based products. The method comprises providing a bite-size carbohydrate-based product and providing personalized guidelines for consuming the bite-size carbohydrate-based product. The bite-size carbohydrate-based product comprises a carbohydrate source comprising a glucogenic:fructogenic carbohydrate ratio ranging between about 1.5 to about 2.5 with at least 60% of the energy content of the bite-size carbohydrate-based product coming from the carbohydrate source. The bite-size carbohydrate-based product further comprises a suitable amount of sodium. The bite-size carbohydrate-based product can also comprise addition components in embodiments as previously discussed.

The personalized guidelines provide the recommended amount (e.g. how many) of the bite-size carbohydrate-based product to consume before exercising, during exercising and/or during recovery after exercising based on one or more characteristics of the athlete and/or one or more training regimens of the athlete. The personal guidelines can also be based on the duration of the training regiment of the athlete.

The bite-size carbohydrate-based products allow for personalized and optimal dosing of carbohydrate for providing energy to the athlete because of the size of the bite-size carbohydrate-based products. For example, the athlete can consume a specified number of bite-size carbohydrate-based products to provide the right amount of energy to the athlete as set forth in the personalized guidelines. In this manner, the portions of the bite-size carbohydrate-based product consumed by the athlete can be easily determined and monitored. In an embodiment, the intake amount (e.g. amount consumed) is guided by the characteristic(s) of the athlete, and the personalized guidelines specify different dosage amounts of the bite-size carbohydrate-based products for the athlete to consume.

To prepare the guidelines and determine what guidelines should be followed, the one or more characteristics of the athlete can be, by way of example, weight, height, age, gender or a combination thereof. Any other physical characteristics of the athlete can also be used for determining the guidelines. The one or more training regimens can include, by way of example, running, swimming, cycling, hiking or any other type of individual sports or group sports (e.g. baseball, football, basketball, soccer, etc.).

The training regimen(s) can be determined based a personal objective of the athlete or consumer of the bite-size carbohydrate-based products. For example, the personal objective can be weight-loss, muscle building, endurance, physical fitness etc.

In an embodiment, the personalized guidelines provide the recommended amount of the bite-size carbohydrate-based product for the athlete to consume during exercising based on at least one of the characteristic of the athlete and the training regimen of the athlete. The size of the bite-size carbohydrate-based product further provides ease of handling and dosing amount determination during the exercising.

The distinct size of the bite-size carbohydrate-based products provides a portability to the bite-size carbohydrate-based products. In addition, as previously discussed, the size also makes it more convenient for the bite-size carbohydrate-based product to be eaten in discrete portion control amounts depending on the guidelines for the athlete. For example, the servings or the amount of the bite-size carbohydrate-based products that the athlete should consume before, during and/or after exercising can be easily adjusted depending on the number of bite-size carbohydrate-based products needed for a specific energy requirement according to the guidelines.

The bite-size carbohydrate-based product can contain a discrete amount of carbohydrates in one or more bite-size products to provide any suitable amount of energy to an athlete. In an embodiment, the carbohydrate amount in the bite-size carbohydrate-based product can be based on a target of about 30 kcal to about 45 kcal in each single bite-size carbohydrate-based product. Moreover, the bite-size carbohydrate-based product can be made so that there is a consistent and countable quantity of calories per single bite-size carbohydrate-based product serving size.

In another embodiment, the present disclosure provides a method of selling a bite-size carbohydrate-based product. The method comprises providing a plurality of bite-size carbohydrate-based products in a package and providing personalized guidelines for consuming the bite-size carbohydrate-based products. Each of the bite-size carbohydrate-based products comprise a carbohydrate source comprising a glucogenic:fructogenic carbohydrate ratio ranging between about 1.5 to about 2.5 with at least 60% of the energy content of the bite-size carbohydrate-based product coming from the carbohydrate source. The bite-size carbohydrate-based product further comprises a suitable amount of sodium. The personalized guidelines provide the recommended amount of the bite-size carbohydrate-based product to consume before and during exercising. The method further comprises selling the bite-size carbohydrate-based product package and the guidelines to an athlete. The personalized guidelines are based on one or more characteristics of the athlete and/or one or more training regimens of the athlete.

In an embodiment, the bite-size carbohydrate-based products are not individually wrapped. In another embodiment, the bite-size carbohydrate-based products are individually wrapped. Preferably, there are between 8 and 10 individual bite-size carbohydrate-based products in each package in one embodiment.

In an alternative embodiment, the present disclosure provides a nutritional kit comprising a plurality of bite-size carbohydrate-based product and personalized guidelines on how many of the bite-size carbohydrate-based products an athlete should consume before and during exercising. A plurality of the bite-size carbohydrate-based products and the personalized guidelines can be kept together in a package.

The personalized guidelines of the nutritional kit can provide the recommended amount of the bite-size carbohydrate-based product to consume before and during exercising based on the body weight or other physical characteristics (e.g. weight, age, gender) of the athlete. In another embodiment, the personalized guidelines of the nutritional kit can provide the recommended amount of the bite-size carbohydrate-based product to the athlete before and during exercising based on the training regimen of the athlete.

In another embodiment, the present disclosure provides a method of enhancing the performance of an athlete. The method comprises providing a bite-size carbohydrate-based product and providing personalized guidelines for consuming the bite-size carbohydrate-based product. The bite-size carbohydrate-based product comprises a carbohydrate source comprising a glucogenic:fructogenic carbohydrate ratio ranging between about 1.5 to about 2.5 with at least 60% of the energy content of the bite-size carbohydrate-based product coming from the carbohydrate source. The bite-size carbohydrate-based product further comprises a suitable amount of sodium. The personalized guidelines provide the recommended amount of the bite-size carbohydrate-based product to consume before the performance based on at least one of a characteristic of the athlete and a training regimen of the athlete.

In an alternative embodiment, the present disclosure provides a method of improving recovery of an athlete after exercising. The method comprises providing a bite-size carbohydrate-based product and providing personalized guidelines for consuming the bite-size carbohydrate-based product. The bite-size carbohydrate-based product comprises a carbohydrate source comprising a glucogenic:fructogenic carbohydrate ratio ranging between about 1.5 to about 2.5 with at least 60% of the energy content of the bite-size carbohydrate-based product coming from the carbohydrate source. The bite-size carbohydrate-based product further comprises a suitable amount of sodium. The personalized guidelines provide the recommended amount of the bite-size carbohydrate-based product to consume during recovery after exercising based on at least one of a characteristic of the athlete and a training regimen of the athlete.

### EXAMPLES

By way of example and not limitation, the following examples are illustrative of various embodiments of the present disclosure.

### Example 1

### Sample Formulation

TABLE 1
Bite-size carbohydrate-based product formulation

| | | Preformed |
|---|---|---|
| | | clusters |
| per 100 g | weight | 100 |
| | kcal | 397 |
| | cal from fat | 7.9% |
| | Carb. | 75.4 |
| | cal from carb | 76.0% |
| | sugars | 47.4 |
| | fiber | 7.4 |
| | protein | 15 |
| | sodium | 191 |
| | fat | 3.5 |
| | saturates | 0.7 |
| | gluc/fruc ratio | 1:5 to 2.5 |
| per | weight | 0.7 |
| cluster | kcal | 2.8 |
| | carb | 0.5 |
| | sugars | 0.3 |
| per | kcal | 200.0 |
| serving | nb of clusters | 72 |

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A bite-size carbohydrate-based product comprising:
a carbohydrate source comprising a glucogenic:fructogenic carbohydrate ratio ranging between about 1.5 to about 2.5 with at least 60% of the energy content of the bite-size carbohydrate-based product coming from the carbohydrate source; and
sodium.

2. The bite-size carbohydrate-based product of claim 1, wherein the sodium ranges from about 200 mg to about 400 mg of sodium per 100 g of the bite-size carbohydrate-based product.

3. The bite-size carbohydrate-based product of claim 1 further comprising a fat, a fiber and a protein.

4. The bite-size carbohydrate-based product of claim 1, wherein the bite-size carbohydrate-based product comprises an energy content of about between about 30 kcal to about 45 kcal.

5. The bite-size carbohydrate-based product of claim 1, wherein the bite-size carbohydrate-based product further comprises a component selected from the group consisting of oat flakes, whole crispies, natural fruits and combinations thereof.

6. The bite-size carbohydrate-based product of claim 1, wherein at least 70% of the energy content of the bite-size carbohydrate-based product comes from the carbohydrates.

7. A method of providing nutrition to an athlete, the method comprising:
providing a bite-size carbohydrate-based product comprising a carbohydrate source comprising a glucogenic:fructogenic carbohydrate ratio ranging between about 1.5 to about 2.5 with at least 60% of the energy content of the bite-size carbohydrate-based product coming from the carbohydrate source, and sodium; and
providing personalized guidelines for consuming the bite-size carbohydrate-based product, wherein the personalized guidelines provide the recommended amount of the bite-size carbohydrate-based product to consume before and during exercising based on at least one of a characteristic of the athlete and a training regimen of the athlete.

8. The method of claim 7, wherein the characteristic of the athlete is selected from the group consisting of weight, height, age, gender and combinations thereof.

9. The method of claim 7, wherein the training regimen is selected from the group consisting of running, swimming, cycling, hiking, football, basketball, baseball, soccer and combinations thereof.

10. The method of claim 7, wherein the personal guidelines are further based on the duration of the training regiment of the athlete.

11. The method of claim 7, wherein training regimen is determined based on a sport.

12. The method of claim 7, wherein training regimen is determined based a personal objective.

13. The method of claim 12, wherein the personal objective is selected from the group consisting of weight-loss, muscle building and combinations thereof.

14. The method of claim 7, wherein the personalized guidelines provide the recommended amount of the bite-size carbohydrate-based product to consume after exercising during recovery based on at least one of the characteristic of the athlete and the training regimen of the athlete.

15. The method of claim 7, wherein the sodium ranges from about 200 mg to about 400 mg of sodium per 100 g of the bite-size carbohydrate-based product.

16. The method of claim 7, wherein the bite-size carbohydrate-based composition further comprises a fat, a fiber and a protein.

17. The method of claim 7, wherein the bite-size carbohydrate-based product comprises an energy content of about 30 kcal to about 45 kcal.

18. The method of claim 7, wherein the bite-size carbohydrate-based product further comprises a component selected from the group consisting of oat flakes, whole crispies, natural fruits and combinations thereof.

19. The method of claim 7, wherein at least 70% of the energy content of the bite-size carbohydrate-based product comes from the carbohydrates.

20. A method of selling a bite-size carbohydrate-based product, the method comprising:
providing a plurality of bite-size carbohydrate-based products in a package, the bite-size carbohydrate-based products comprising a carbohydrate source comprising a glucogenic:fructogenic carbohydrate ratio ranging between about 1.5 to about 2.5 with at least 60% of the energy content of the bite-size carbohydrate-based product coming from the carbohydrate source, and sodium;
providing personalized guidelines for consuming the bite-size carbohydrate-based products, wherein the personalized guidelines provide the recommended amount of the bite-size carbohydrate-based product to consume before and during exercising; and
selling the bite-size carbohydrate-based product package and the guidelines to an athlete.

21. The method of claim 20, wherein the personalized guidelines are based on at least one of a characteristic of the athlete and a training regimen of the athlete.

22. A nutritional kit comprising a plurality of bite-size carbohydrate-based products and personalized guidelines on how many of the bite-size carbohydrate-based products an athlete should consume before and during exercising, the bite-size carbohydrate-based products comprising a carbohydrate source comprising a glucogenic:fructogenic carbohydrate ratio ranging between about 1.5 to about 2.5 with at least 60% of the energy content of the bite-size carbohydrate-based product coming from the carbohydrate source, and sodium.

23. The nutritional kit of claim 22, wherein a plurality of the bite-size carbohydrate-based products and the personalized guidelines are together in a package.

24. The nutritional kit of claim 22, wherein the personalized guidelines provide the recommended amount of the bite-size carbohydrate-based product to consume before and during exercising based on a physical characteristic of the athlete.

25. The nutritional kit of claim 22, wherein the personalized guidelines provide the recommended amount of the bite-size carbohydrate-based product to the athlete before and during exercising based on the training regimen of the athlete.

26. A method of enhancing the performance of an athlete, the method comprising:
providing a bite-size carbohydrate-based product comprising a carbohydrate source comprising a glucogenic:fructogenic carbohydrate ratio ranging between about 1.5 to about 2.5 with at least 60% of the energy content of the bite-size carbohydrate-based product coming from the carbohydrate source, and sodium; and
providing personalized guidelines for consuming the bite-size carbohydrate-based product, wherein the personalized guidelines provide the recommended amount of the bite-size carbohydrate-based product to consume before the performance based on at least one of a characteristic of the athlete and a training regimen of the athlete.

27. The method of claim 26, wherein the characteristic of the athlete is selected from the group consisting of weight, height, age, gender and combinations thereof.

28. A method of improving recovery of an athlete after exercising, the method comprising:
providing a bite-size carbohydrate-based product comprising a carbohydrate source comprising a glucogenic:fructogenic carbohydrate ratio ranging between about 1.5 to about 2.5 with at least 60% of the energy content of the bite-size carbohydrate-based product coming from the carbohydrate source, and sodium; and
providing personalized guidelines for consuming the bite-size carbohydrate-based product, wherein the personalized guidelines provide the recommended amount of the bite-size carbohydrate-based product to consume during recovery after exercising based on at least one of a characteristic of the athlete and a training regimen of the athlete.

29. The method of claim 28, wherein the characteristic of the athlete is selected from the group consisting of weight, height, age, gender and combinations thereof.
